(19) United States
(12) Patent Application Publication
(10) Pub. No.: US 2025/0139946 A1
(43) Pub. Date: May 1, 2025

(54) INFORMATION PROCESSING DEVICE, INFORMATION PROCESSING METHOD, AND PROGRAM

(71) Applicant: SONY GROUP CORPORATION, TOKYO (JP)

(72) Inventors: SHO INAYOSHI, TOKYO (JP); SATOSHI OZAKI, TOKYO (JP); JUNJI OTSUKA, TOKYO (JP); SOTA SHOMAN, TOKYO (JP)

(21) Appl. No.: 18/836,093

(22) PCT Filed: Jan. 30, 2023
(86) PCT No.: PCT/JP2023/002805

§ 371 Date: Aug. 6, 2024

(30) Foreign Application Priority Data
Feb. 15, 2022 (JP) 2022-021338

(51) Int. Cl. G06V 10/774; G06T 17/00

(52) U.S. Cl. CPC G06V 10/774; G06T 17/00

(57) ABSTRACT

The present disclosure relates to an information processing device, an information processing method, and a program for enabling update and use of a surgical tool recognizer after confirming that the surgical tool recognizer is in a state of being capable of recognizing a new surgical tool. Computer graphics (CG) data serving as learning data to be used to learn a recognizer that recognizes a new surgical tool is generated on the basis of an image including the new surgical tool, the recognizer that recognizes the new surgical tool is caused to learn on the basis of the CG data, an evaluation value that evaluates a recognition result of the new surgical tool by the recognizer based on the image including the new surgical tool is calculated, and the learning of the recognizer based on the CG data is continued according to the evaluation value. The present disclosure can be applied to a surgical image display system.

### TECHNICAL FIELD

The present disclosure relates to an information processing device, an information processing method, and a program, and particularly relates to an information processing device, an information processing method, and a program for updating a surgical tool recognizer to become usable after confirming that the surgical tool recognizer is in a state of being capable of recognizing a new surgical tool.

### BACKGROUND ART

As a medical support technology based on a surgical image, there has been proposed a technology in which a surgical tool, which is a tool used for surgery, is recognized on the basis of the surgical image and support based on a recognition result is performed.

Here, as a technique for recognizing the surgical tool, a technique related to a surgical tool recognizer that recognizes the surgical tool from the surgical image, which is realized by machine learning such as deep neural network (DNN), is expected as a technique responsible for medical support.

The surgical tool recognizer is realized by learning based on a learning model including a learning data set using the surgical tool used in advance.

However, in general, the surgical tools having the same function do not completely match each environment (hospital) to be introduced.

Therefore, there is a case where the surgical tool recognizer cannot appropriately recognize a new surgical tool due to an influence of the introduction environment or the like, and it is necessary to generate a corresponding new learning model and perform learning using the new learning model each time the new surgical tool appears.

Therefore, a technique for creating a learning data set necessary for recognizing a new object has been proposed (see Patent Document 1).

### CITATION LIST

### Patent Document

Patent Document 1: Japanese Patent Application Laid-Open No. 2021-047839

### SUMMARY OF THE INVENTION

### Problems to be Solved by the Invention

However, even if the new learning model corresponding to the new surgical tool is generated by the technique described in Patent Document 1 and the surgical tool recognizer is learned with the new learning model, there is no method of confirming whether or not the new surgical tool can be actually recognized after the surgical tool recognizer is learned and updated.

The present disclosure has been made in view of such a situation, and in particular enables update and use of a surgical tool recognizer after confirming that the surgical tool recognizer becomes in a state of being capable of sufficiently recognizing a new surgical tool without being affected by an introduction environment.

### Solutions to Problems

An information processing device and a program according to one aspect of the present disclosure are an information processing device including: a computer graphics (CG) data generation unit configured to generate, on a basis of an image including a new surgical tool, CG data serving as learning data to be used to learn a recognizer that recognizes the new surgical tool; a learning unit configured to cause the recognizer to learn, the recognizer recognizing the new surgical tool, on a basis of the CG data; and an evaluation unit configured to calculate an evaluation value that evaluates a recognition result of the new surgical tool by the recognizer based on the image, in which the learning unit continues the learning of the recognizer according to the evaluation value, and a program.

An information processing method according to one aspect of the present disclosure is an information processing method including steps of: generating, on a basis of an image including a new surgical tool, computer graphics (CG) data serving as learning data to be used to learn a recognizer that recognizes the new surgical tool; causing the recognizer to learn, the recognizer recognizing the new surgical tool, on a basis of the CG data; and calculating an evaluation value that evaluates a recognition result of the new surgical tool by the recognizer based on the image, in which the learning of the recognizer is continued according to the evaluation value.

In one aspect of the present disclosure, computer graphics (CG) data serving as learning data to be used to learn a recognizer that recognizes a new surgical tool is generated on the basis of an image including the new surgical tool, the recognizer that recognizes the new surgical tool is learned on the basis of the CG data, an evaluation value that evaluates a recognition result of the new surgical tool by the recognizer based on the image is calculated, and the learning of the recognizer is continued according to the evaluation value.

### BRIEF DESCRIPTION OF DRAWINGS

FIG. 1 is a diagram for describing a method of learning a surgical tool recognizer.

FIG. 2 is a diagram for describing a recognition result when learning of the surgical tool recognizer is insufficient.

FIG. 3 is a diagram for describing an example in which a 3DCG model is different from a surgical image of a surgical tool.

FIG. 4 is a diagram for describing an outline of the present disclosure.

FIG. 5 is a diagram for describing a configuration example of a surgical image display system of the present disclosure.

FIG. 6 is a diagram for describing a display example of a feedback image.

FIG. 7 is a diagram for describing a method of acquiring three-dimensional point group information of a new surgical tool.

FIG. 8 is a diagram for describing a method of generating a 3DCG model from the three-dimensional point group information.

FIG. 9 is a diagram for describing a method of learning a new surgical tool registered recognizer.

FIG. 10 is a diagram for describing a method of recreating a 3DCG model.

FIG. 11 is a diagram for describing a method of calculating a stability evaluation value.

FIG. 12 is a diagram for describing a method of calculating a consistency evaluation value.

FIG. 13 is a flowchart for describing new surgical tool registration processing.

FIG. 14 is a flowchart for describing a modification of the new surgical tool registration processing.

FIG. 15 illustrates a configuration example of a general-purpose computer.

### MODE FOR CARRYING OUT THE INVENTION

Hereinafter, a preferred embodiment of the present disclosure will be described in detail with reference to the accompanying drawings. Note that, in the present specification and the drawings, components having substantially the same functional configurations are denoted by the same reference signs, and redundant descriptions are omitted.

Hereinafter, a mode for carrying out the present technology will be described. Description will be given in the following order.
1. Outline of Present Disclosure
2. Favorable Embodiment
3. Modification of New Surgical Tool Registration Processing
4. Example of Execution by Software

### 1. Outline of Present Disclosure

The present disclosure in particular enables update and use of a surgical tool recognizer after confirming that the surgical tool recognizer is in a state of being capable of sufficiently recognizing a new surgical tool.

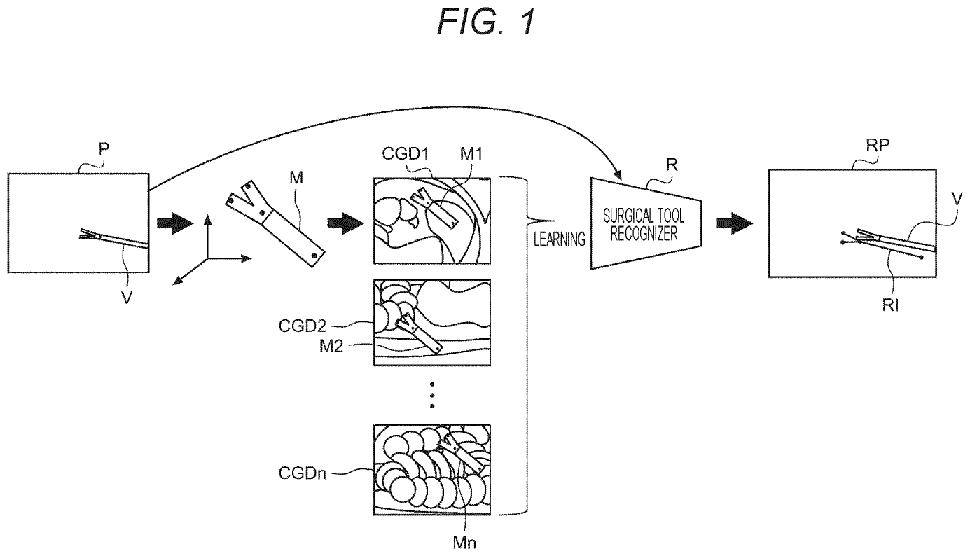

First, an outline of the present disclosure will be described with reference to FIG. 1.

The present disclosure enables, for a surgical tool recognizer that recognizes a surgical tool that is a tool for surgery captured in a surgical image by a laparoscope or the like, update and use of the surgical tool recognizer after confirming that the surgical tool recognizer becomes in a state of being capable of recognizing the new surgical tool in updating the surgical tool recognizer to be able to recognize the new surgical tool.

Here, a learning procedure of the surgical tool recognizer that enables registration and recognition of the new surgical tool will be briefly described.

As a first step, as illustrated in the left part of FIG. 1, a three-dimensional computer graphics (3DCG) model M of the new surgical tool is generated by a structure from motion to be described below on the basis of a plurality of intraperitoneal images P obtained by imaging a real image V of the new surgical tool with a laparoscope or the like while changing the real image V to various angles.

As a second step, as illustrated in the central part of FIG. 1, a plurality of background images assumed to be captured by the laparoscope is generated, and a situation actually used by simulation using the 3DCG model M of the new surgical tool and the plurality of background images are combined to generate computer graphics (CG) data CGD1 to CGDn serving as learning data.

As a third step, a surgical tool recognizer R is learned by learning using the computer graphics (CG) data CGD1 to CGDn.

By the learning processing as described above, the learned surgical tool recognizer R recognizes the new surgical tool from the intraperitoneal image P, and outputs a recognition result image RP obtained by superimposing and displaying a recognition result RI including a CG image on the real image V.

However, if the learning of the surgical tool recognizer R is insufficient, a recognition position of the recognition result RI is not stabilized between frames, and for a user, the position of the recognition result RI seems to be unsteadily changing with respect to the real image V.

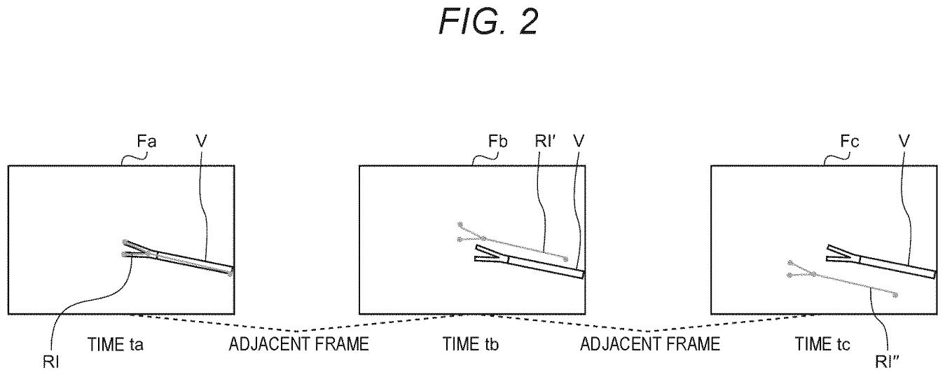

That is, for example, as indicated by respective recognition results RI, RI′, and RI″ in frames Fa to Fc adjacent in a time direction of the time ta to tc in FIG. 2, the stability of each position is low, and the each position seems to be unsteadily and unstably changing with respect to the position of the real image V.

Furthermore, if the 3DCG model M for the new surgical tool has a large deviation from the recognition result RI, that is, if the consistency between the 3DCG model M and the recognition result RI is low, CG data generated using the current 3DCG model M cannot function as appropriate learning data, and there is a possibility that recognition accuracy cannot be efficiently improved even if the learning is continued.

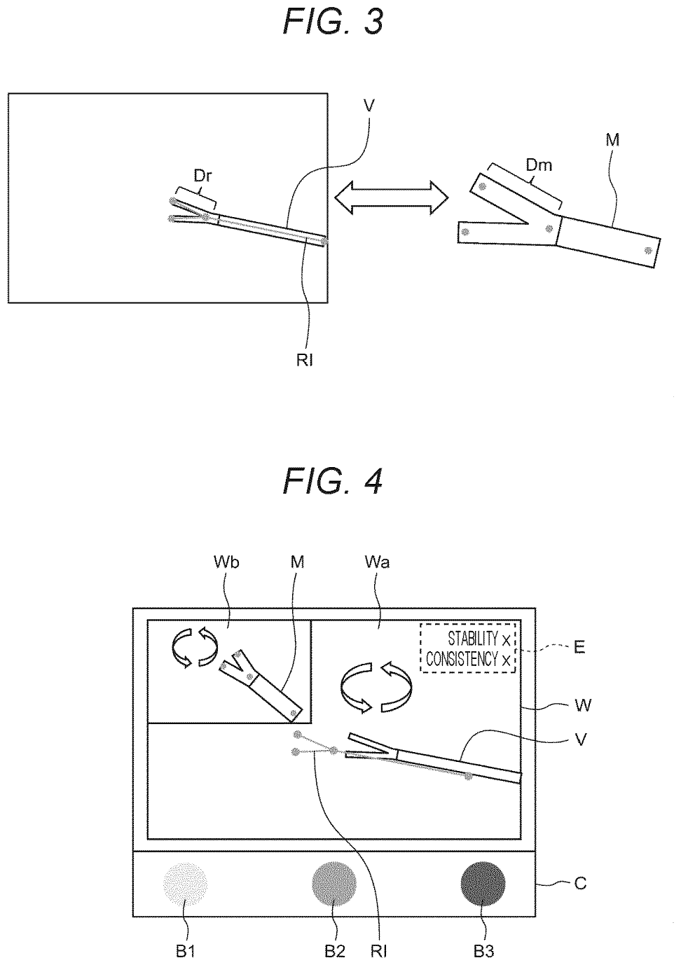

That is, in a case where the 3DCG model M in the right part is thicker than the recognition result RI illustrated in the left part of FIG. 3, and a length Dr of the recognition result RI from a fulcrum part to a distal end is different from a corresponding length Dm in the 3DCG model M, there is a possibility that the generated CG data does not function as appropriate learning data.

Therefore, in the present disclosure, when the surgical tool recognizer that recognizes the surgical tool in the surgical image is updated so as to become able to recognize the new surgical tool, information indicating the degree of recognition of the new surgical tool by the surgical tool recognizer is presented to the user as a feedback image.

When it is determined that the degree of recognition is low and the learning is insufficient on the basis of the information indicating the degree of recognition of the new surgical tool by the surgical tool recognizer presented in the feedback image, the user causes the imaging and learning of the new surgical tool to be continued in the introduction environment.

Since the information indicating the degree of recognition of the new surgical tool by the surgical tool recognizer is presented as the feedback image even while the learning is continued, how much the degree of recognition of the new surgical tool by the surgical tool recognizer is improved with the continuation of the learning is presented to the user as needed.

Then, when the user considers that the degree of recognition of the new surgical tool by the surgical tool recognizer presented as the feedback image is sufficient, the user terminates the learning and updates and uses the surgical tool recognizer.

Therefore, when the surgical tool recognizer is updated so as to be able to recognize the new surgical tool, it is possible to prevent a trouble that occurs due to a fact that the new surgical tool is not recognized during actual surgery.

More specifically, the surgical tool recognizer is constructed by learning based on learning data (computer graphics (CG) data) including a composite image generated by combining the 3DCG model of the new surgical tool to be recognized and various intraperitoneal images by simulation.

Therefore, in the present disclosure, the feedback image including information for qualitatively and quantitatively evaluating the degree of recognition of the new surgical tool by the surgical tool recognizer is generated and presented to the user, thereby causing the user to recognize the degree of recognition of the new surgical tool.

The user determines whether or not the degree of recognition of the new surgical tool by the surgical tool recognizer is sufficient on the basis of the information for qualitatively and quantitatively evaluating the degree of recognition of the new surgical tool by the surgical tool recognizer presented as the feedback image.

Here, in the case where the user regards that the degree of recognition of the new surgical tool by the surgical tool recognizer is low and the learning is insufficient, the degree of recognition of the new surgical tool by the surgical tool recognizer is improved by continuing the learning using learning data (CG data).

However, in a case where the 3DCG model of the new surgical tool is not appropriately formed, there is a possibility that the degree of recognition by the surgical tool recognizer cannot be effectively improved even if the learning data is randomly increased and the learning is continued.

Therefore, the 3DCG model for the current new surgical tool and the actual recognition result of the new surgical tool are presented to the user so that the user can make a comparison, and allow the user to qualitatively determine whether or not the 3DCG model of the current new surgical tool is appropriate.

Therefore, in the case where it is regarded by the judgement of the user that the 3DCG model of the new surgical tool does not sufficiently match the recognition result of the new surgical tool, the degree of recognition is improved by recreating the 3DCG model on the basis of the current recognition result and then generating the learning data (CG data), and continuing the learning.

More specifically, by generating the feedback image as illustrated in FIG. 4 and presenting the feedback image to the user, the degree of recognition of the new surgical tool by the surgical tool recognizer is presented.

FIG. 4 is a diagram for describing an outline of an external appearance configuration example of a surgical image display device to which the technology of the present disclosure is applied.

The surgical image display device in FIG. 4 includes a display unit W and an operation unit C. The display unit W displays the feedback image that presents the information indicating the degree of recognition of the new surgical tool by the surgical tool recognizer described above.

The feedback image displayed on the display unit W in FIG. 4 includes an in-operative field image display unit Wa and a model display unit Wb. The in-operative field image display unit Wa displays a surgical image in an operative field captured by the unillustrated laparoscope. The model display unit Wb displays the 3DCG model M used for learning the surgical tool recognizer.

In the in-operative field image display unit Wa of FIG. 4, the real image V of the surgical tool in the surgical image captured by the unillustrated laparoscope or the like and the recognition result RI by the surgical tool recognizer are superimposed and displayed.

Since a gap between the real image V of the surgical tool and the recognition result RI by the surgical tool recognizer is displayed with the display of the in-operative field image display unit Wa, the user can qualitatively recognize the degree of recognition by the surgical tool recognizer.

Furthermore, an evaluation display unit E is provided in the in-operative field image display unit Wa, and the evaluation for the recognition result RI such as the stability between frames with respect to the recognition result RI and the consistency between the 3DCG model M and the recognition result RI, that is, information for quantitatively evaluating the degree of recognition by the surgical tool recognizer is displayed.

With the display of the evaluation display unit E, the user can determine the degree of recognition of the new surgical tool by the surgical tool recognizer on the basis of the quantitative evaluation.

Furthermore, the 3DCG model M of the new surgical tool used for learning the surgical tool recognizer is displayed in the model display unit Wb in FIG. 4.

With the display of the model display unit Wb, the user can compare and view the real image V of the surgical tool, the recognition result RI, and the 3DCG model M in the in-operative field image display unit Wa, and thus can qualitatively recognize the consistency between the current 3DCG model M and the recognition result RI of the actual surgical tool.

The operation unit C is provided with a learning continuation button B1 operated when continuing the learning, a learning termination button B2 operated when terminating the learning, and a 3DCG model recreation button B3 operated when recreating the 3DCG model.

When the user operates the operation unit C according to display content of the in-operative field image display unit Wa and the model display unit Wb in the feedback image displayed on the display unit W, continuation of the learning, termination of the learning, or recreation of the 3DCG model of the new surgical tool recognizer can be executed.

That is, in the in-operative field image display unit Wa in FIG. 4, the real image V of the surgical tool and the recognition result RI are displayed in a shifted state, and cross marks indicating that both the stability and the consistency in the evaluation display unit E are not appropriate are attached.

In the case of such display, the user can determine that the surgical tool recognizer cannot sufficiently recognize the new surgical tool, that is, the learning of the new surgical tool recognizer is insufficient, and thus operates, for example, the learning continuation button B1.

Furthermore, in the model display unit Wb of FIG. 4, the 3DCG model M is expressed thicker than the real image V of the new surgical tool, and also the lengths of various parts of the 3DCG model M and the recognition result RI are different. In the case of such display, the user can determine that the consistency between the 3DCG model M of the new surgical tool and the recognition result RI is low, and thus operates, for example, the 3DCG model recreation button B3.

Therefore, the 3DCG model M is recreated on the basis of the current recognition result RI, and thus is recreated in a state close to the recognition result RI, and the CG data using the recreated 3DCG model M is created as the learning data, and the learning of the surgical tool recognizer is continued.

That is, in any case, since the feedback image is continuously displayed on the display unit W while the learning is continued, the state where the degree of recognition of the new surgical tool is improved by continuing the learning is presented in the feedback image.

Then, for example, when the real image V of the new surgical tool and the recognition result RI are superimposed and displayed at a matching position, the 3DCG model M and the recognition result RI are displayed as having sufficient consistency, and the information indicating that both the stability and the consistency in the evaluation display unit E are appropriate is displayed on the basis of the display content of the in-operative field image display unit Wa and the model display unit Wb of the feedback image displayed on the display unit W, it is regarded that the sufficient learning has been performed enough to the state where the new surgical tool recognizer can recognize the new surgical tool.

In such a case, the user operates the learning termination button B2 to terminate the learning, and updates and uses the surgical tool recognizer learned for the new surgical tool.

Furthermore, in the present disclosure, when the evaluation is lower than a predetermined value on the basis of the quantitative evaluation for the recognition result RI such as the stability between frames with respect to the recognition result RI and the consistency between the 3DCG model M and the recognition result RI, the learning is continued or the 3DCG model M is recreated without waiting for the user's operation.

As a result, according to the present disclosure, in the case of updating the surgical tool recognizer to be able to recognize the new surgical tool, the user can update and use the surgical tool recognizer after confirming that the surgical tool recognizer can sufficiently recognize the new surgical tool.

### 2. Favorable Embodiment

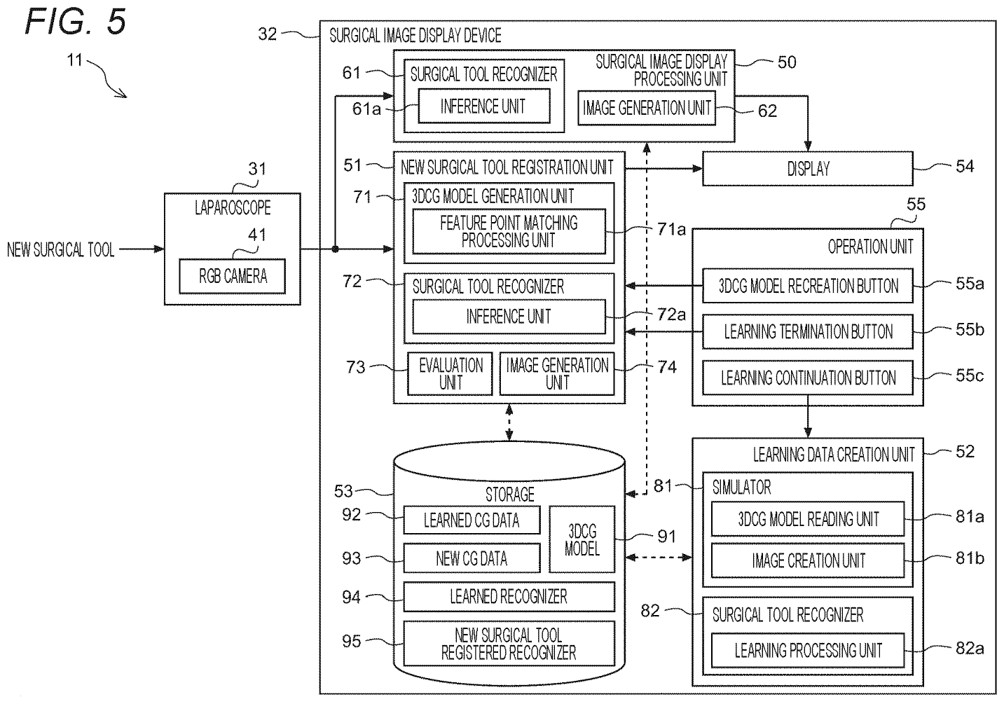

Next, a configuration example of a favorable embodiment of the surgical image display system to which the technology of the present disclosure is applied will be described with reference to FIG. 5.

A surgical image display system 11 in FIG. 5 captures an image of the operative field in an abdominal cavity, recognizes the surgical tool included in the captured image, and displays the CG image of the surgical tool as the recognition result to be superimposed on the surgical tool in the image.

The surgical image display system 11 includes the surgical tool recognizer that recognizes the surgical tool included in the image by the image, generates the CG image representing the surgical tool recognized by the surgical tool recognizer as the recognition result, and superimposes and displays the CG image on the surgical tool in the image.

The surgical tool recognizer is a recognizer including, for example, a deep neural network (DNN) or the like formed by machine learning using an image of an operative field including a surgical tool as the learning data. Therefore, to enable the surgical tool recognizer to recognize the new surgical tool, it is necessary to insert the new surgical tool into the abdominal cavity, cause an RGB camera 41 of a laparoscope 31 to capture an image, and perform learning processing based on the captured image.

In the learning processing for causing the surgical tool recognizer to recognize the new surgical tool, the surgical image display system 11 generates the feedback image indicating the degree of recognition of the new surgical tool by the surgical tool recognizer and presents the feedback image to the user.

The user confirms the degree of recognition of the new surgical tool by the surgical tool recognizer by viewing the feedback image presented by the surgical image display system 11, and continues the learning when determining that the degree of recognition is insufficient. Then, when determining that the learning is sufficient, the user terminates the learning of the surgical tool recognizer, and updates and use the surgical tool recognizer.

More specifically, the surgical image display system 11 includes the laparoscope 31 and a surgical image display device 32.

The laparoscope 31 includes the RGB camera 41, captures an image of the operative field including the surgical tool in the abdominal cavity, and outputs the captured image to the surgical image display device 32 as a surgical image.

The RGB camera 41 includes, for example, a complementary metal oxide semiconductor (CMOS) image sensor or a charge-coupled device (CCD) image sensor, captures the image of the operative field in the abdominal cavity serving as an imaging field of view, and outputs the image as image data to the surgical image display device 32.

When executing the learning processing of the surgical tool recognizer for the new surgical tool, the laparoscope 31 images the new surgical tool from various angles by changing the new surgical tool to various angles and outputs the captured images to the surgical image display device 32.

When executing the learning processing of the surgical tool recognizer for the new surgical tool, the laparoscope 31 may capture the image of the new surgical tool in an environment other than the surgical environment. However, to improve recognition accuracy in the surgical environment, it is desirable to capture the image in the surgical environment.

The surgical image display device 32 includes the surgical tool recognizer, recognizes the surgical tool in the image on the basis of the image supplied from the laparoscope 31, and superimposes and displays the CG image for representing the recognized surgical tool on the real image of the surgical tool in the image.

The surgical image display device 32 executes learning processing (new surgical tool registration processing) of the surgical tool recognizer for recognizing the new surgical tool on the basis of the image including the new surgical tool.

More specifically, the surgical image display device 32 includes a surgical image display processing unit 50, a new surgical tool registration unit 51, a learning data creation unit 52, a storage 53, a display 54, and an operation unit 55.

The surgical image display processing unit 50 recognizes the surgical tool on the basis of the image of the operative field including the surgical tool captured by the RGB camera 41 of the laparoscope 31, generates an image obtained by superimposing the CG image representing the surgical tool as the recognition result on the surgical tool in the image, and displays the image on the display 54.

At this time, the surgical image display processing unit 50 reads a learned recognizer 94 learned in a state of being capable of recognizing the new surgical tool from the storage 53, and recognizes the surgical tool on the basis of the image of the operative field including the surgical tool captured by the RGB camera 41 of the laparoscope 31.

That is, the surgical image display processing unit 50 executes surgical image display processing after the learned recognizer 94 learned enough to the state of being capable of recognizing the new surgical tool is registered in the storage 53, recognizes the surgical tool, and displays the surgical image obtained by superimposing the CG image serving as the recognition result on the surgical tool on the display 54.

More specifically, the surgical image display processing unit 50 includes a surgical tool recognizer 61 and an image generation unit 62.

The surgical tool recognizer 61 includes an inference unit 61a, reads the learned recognizer 94 stored in the storage 53, recognizes (infers) the surgical tool on the basis of the image supplied from the laparoscope 31, and supplies the recognition result to the image generation unit 62.

The image generation unit 62 generates an image obtained by superimposing the CG image of the surgical tool serving as the recognition result supplied from the surgical tool recognizer 61 on the surgical tool in the image supplied from the laparoscope 31, and displays the image on the display 54.

When generating a new surgical tool recognizer for registering the new surgical tool, the new surgical tool registration unit 51 generates a new surgical tool registered recognizer 95 by learning on the basis of the new surgical tool in the image supplied from the laparoscope 31 and stores the same in the storage 53.

More specifically, the new surgical tool registration unit 51 includes a 3DCG model generation unit 71, a surgical tool recognizer 72, an evaluation unit 73, and an image generation unit 74.

The 3DCG model generation unit 71 generates the 3DCG model corresponding to the new surgical tool in the image on the basis of the image supplied from the laparoscope 31, and stores the 3DCG model as a 3DCG model 91 in the storage 53.

More specifically, the 3DCG model generation unit 71 includes a feature point matching processing unit 71a, executes structure from motion processing by matching feature points in a region of the surgical tool between frames on the basis of images of the new surgical tool captured at various angles supplied from the laparoscope 31, generates three-dimensional point group information of the new surgical tool, and generates the 3DCG model of the new surgical tool using the generated three-dimensional point group information.

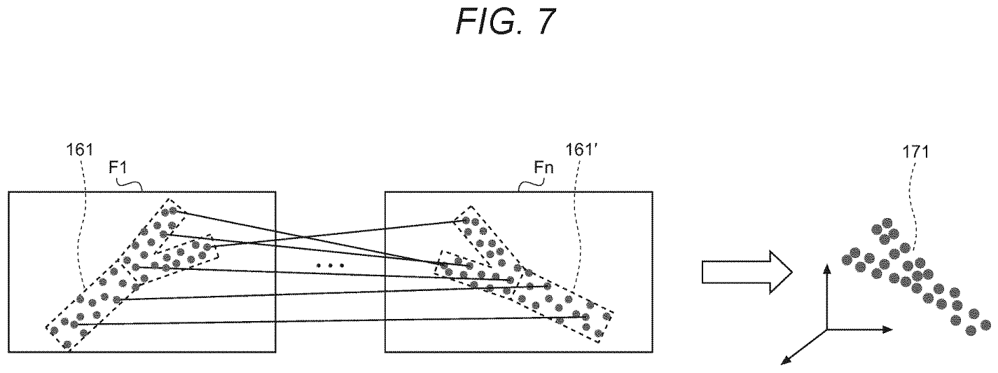

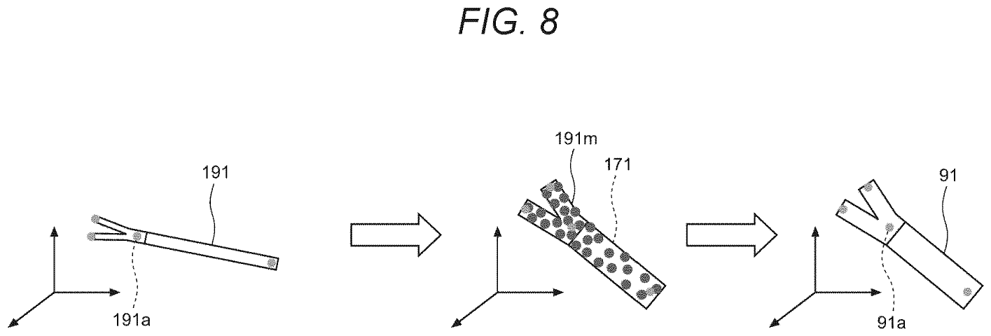

Note that details of the method of generating the 3DCG model 91 by the 3DCG model generation unit 71 will be described below with reference to FIGS. 7 and 8.

The surgical tool recognizer 72 has a configuration corresponding to the surgical tool recognizer 61 of the surgical image display processing unit 50, and includes an inference unit 72a. When the new surgical tool registered recognizer 95 is generated by the learning data creation unit 52 on the basis of the 3DCG model 91, the surgical tool recognizer 72 reads the new surgical tool registered recognizer 95.

Then, the surgical tool recognizer 72 controls the inference unit 72a to cause the new surgical tool registered recognizer 95 to function, recognizes the new surgical tool on the basis of the image supplied from the laparoscope 31, generates the CG image corresponding to the new surgical tool serving as the recognition result, and outputs the CG image to the evaluation unit 73 and the image generation unit 74.

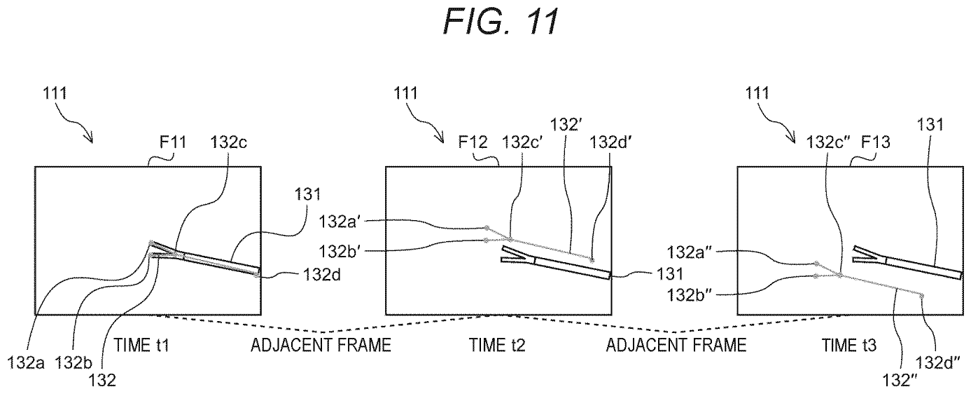

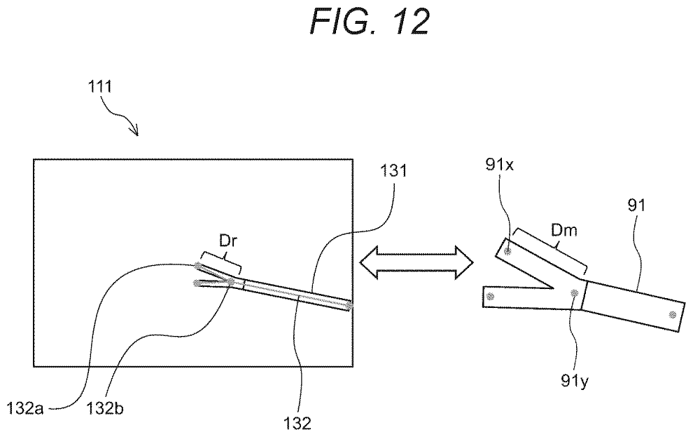

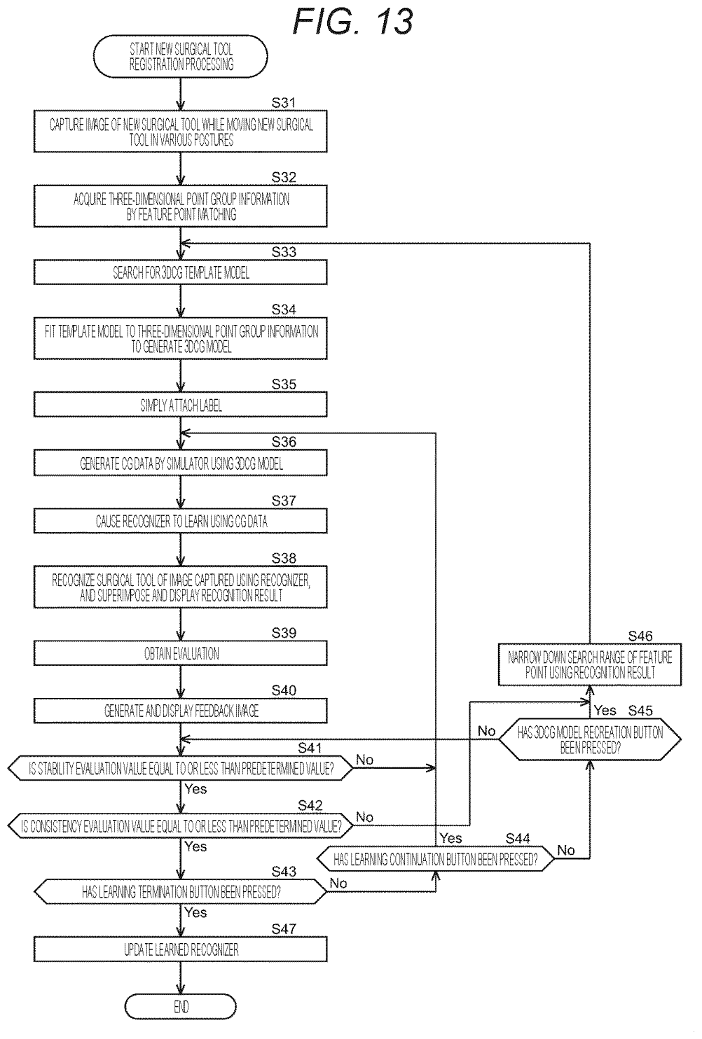

The evaluation unit 73 generates information that evaluates the recognition result on the basis of the CG image that is the recognition result of the surgical tool recognizer 72 and the 3DCG model 91, and supplies the information to the image generation unit 74. Note that details of the evaluation of the recognition result will be described below with reference to FIGS. 11 and 12.

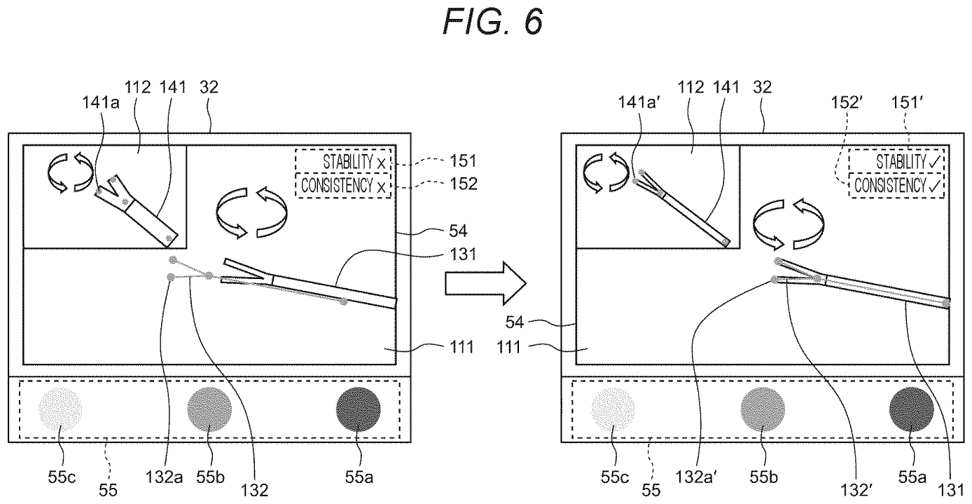

The image generation unit 74 combines the image obtained by superimposing and displaying the CG image as the recognition result on the new surgical tool in the image supplied from the laparoscope 31 with a display image of the 3DCG model 91, further generates the feedback image obtained by adding the evaluation result supplied from the evaluation unit 73 on the superimposed image, and displays the feedback image on the display 54. The feedback image will be described below with reference to FIG. 6.

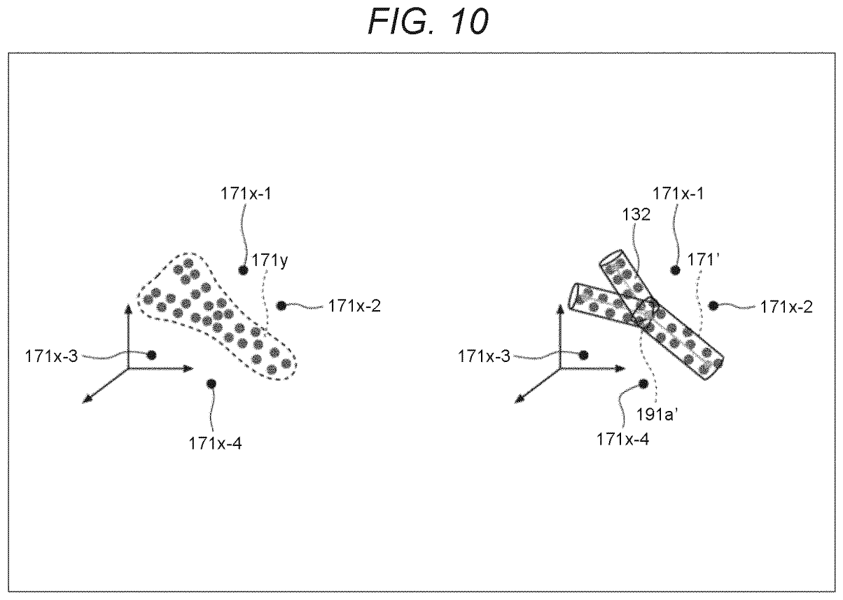

When the operation unit 55 is operated by the user on the basis of the feedback image displayed on the display 54 and the recreation of the 3DCG model for the new surgical tool is instructed, the 3DCG model generation unit 71 recreates the 3DCG model on the basis of the current recognition result and stores the same in the storage 53 as the 3DCG model 91. Note that details of the recreation of the 3DCG model will be described below with reference to FIG. 10.

Furthermore, when the operation unit 55 is operated by the user on the basis of the feedback image displayed on the display 54 and the termination of the learning is instructed, the new surgical tool registration unit 51 terminates the learning of the current new surgical tool registered recognizer 95, and updates the learned recognizer 94 with the new surgical tool registered recognizer 95 so as to enable the surgical image display processing unit 50 to use the learned recognizer 94.

When the new surgical tool registered recognizer 95 is learned by the new surgical tool registration unit 51 and new 3DCG model 91 is registered, the learning data creation unit 52 reads the new 3DCG model 91 and generates the CG data serving as the learning data by simulation, and stores the generated CG data in the storage 53 as new CG data 93.

Furthermore, the learning data creation unit 52 generates the new surgical tool registered recognizer 95 by learning the learned recognizer 94 using the new CG data 93 and learned CG data 92 registered in the storage 53, and stores the same in the storage 53.

More specifically, the learning data creation unit 52 includes a simulator 81 and a surgical tool recognizer 82.

The simulator 81 includes a 3DCG model reading unit 81a and an image creation unit 81b, and controls the 3DCG model reading unit 81a to read the new 3DCG model 91 from the storage 53 when the new 3DCG model 91 is registered in the storage 53.

Then, the simulator 81 controls the image creation unit 81b to generate various images of the operative field, and generates new CG data 93 serving as the learning data by performing CG synthesis of the generated image of the operative field and the 3DCG model 91, and stores the same in the storage 53.

The surgical tool recognizer 82 includes a learning processing unit 82a, and controls the learning processing unit 82a to generate the new surgical tool registered recognizer 95 by the learning processing using the learned CG data 92, the new CG data 93, and the learned recognizer 94 stored in the storage 53.

Note that details of the processing of generating the new surgical tool registered recognizer 95 by the learning processing using the learned CG data 92, the new CG data 93, and the learned recognizer 94 by the learning processing unit 82a will be described below with reference to FIG. 6.

The display 54 is a display device including a liquid crystal display (LCD) or organic electro-luminescence (EL), and displays the surgical image supplied from the surgical image display processing unit 50 and the feedback image supplied from the new surgical tool registration unit 51.

The operation unit 55 includes operation buttons to which user's instructions based on the feedback image displayed on the display 54 are input when the learning processing (new surgical tool registration processing) for registering the new surgical tool is performed, and an operation signal corresponding to an operation of the corresponding button is supplied to at least one of the new surgical tool registration unit 51 or the learning data creation unit 52.

More specifically, the operation unit 55 includes a 3DCG model recreation button 55a, a learning termination button 55b, and a learning continuation button 55c.

The 3DCG model recreation button 55a is a button operated when the user determines that the recreation of the 3DCG model is necessary on the basis of the feedback image displayed on the display 54.

When the 3DCG model recreation button 55a is operated, the 3DCG model generation unit 71 recreates the 3DCG model 91 on the basis of the current recognition result. Furthermore, in a case where a consistency evaluation value obtained by the evaluation unit 73 is lower than a predetermined value, the 3DCG model generation unit 71 recreates the 3DCG model 91 on the basis of the current recognition result.

The learning termination button 55b is a button operated when the user determines that the new surgical tool registered recognizer 95 can sufficiently recognize the new surgical tool on the basis of the feedback image displayed on the display 54.

When the learning termination button 55b is operated, the surgical tool recognizer 82 in the learning data creation unit 52 terminates the learning processing of the new surgical tool registered recognizer 95 by the learning processing unit 82a, and updates the learned recognizer 94 with the new surgical tool registered recognizer 95 so as to enable the surgical image display processing unit 50 to use the learned recognizer 94.

The learning continuation button 55c is a button operated when the user determines that the degree of recognition of the new surgical tool by the new surgical tool registered recognizer 95 is low, the learning is regarded to be insufficient, and continuous learning is necessary on the basis of the feedback image displayed on the display 54.

When the learning continuation button 55c is operated, the surgical tool recognizer 82 in the learning data creation unit 52 continues the learning processing of the new surgical tool registered recognizer 95 by the learning processing unit 82a, further using more CG data that is the learning data generated by the simulator 81.

### <Display Example of Feedback Image>

Next, an external appearance configuration example of the surgical image display device 32 in FIG. 5 will be described, and display examples of the feedback image will be described with reference to FIG. 6.

FIG. 6 illustrates an external appearance configuration example of the surgical image display device 32 in FIG. 5. The display 54 is provided in the upper part and the operation unit 55 is provided in the lower part of the surgical image display device 32 in FIG. 6.

FIG. 6 illustrates display examples of two feedback images that present the degrees of recognition of the above-described new surgical tool by the surgical tool recognizer on the display 54.

The left part of FIG. 6 illustrates a display example of the feedback image in the state where the degree of recognition of the new surgical tool by the surgical tool recognizer is insufficient, and the right part of FIG. 6 illustrates a display example of the feedback image in the state where the degree of recognition of the new surgical tool by the surgical tool recognizer is sufficient.

The feedback image displayed on the display 54 in FIG. 6 includes an image display unit 111 and a model display unit 112. The image display unit 111 displays an image captured by the laparoscope 31. The model display unit 112 displays the 3DCG model used for learning the surgical tool recognizer.

In the image display unit 111, a real image 131 of the surgical tool in the image captured by the laparoscope 31 and a CG image 132 serving as the recognition result by the surgical tool recognizer are superimposed and displayed.

A label representing a part is attached to the CG image 132 including the recognition result, a linear portion in the drawing represents that a shaft or a frame is labeled, and a point represents a movable fulcrum or a distal end.

With the display of the image display unit 111, the gap between the real image 131 of the surgical tool and the CG image 132 including the recognition result by the surgical tool recognizer is displayed.

That is, in the case where the degree of recognition is insufficient, the position of the CG image 132 including the recognition result being shifted from the real image 131 is expressed, as illustrated in the left part of FIG. 6.

In contrast, in the case where the degree of recognition is sufficient, a CG image 132′ including the recognition result matching the real image 131 is expressed as illustrated in the right part of FIG. 6.

With the display of the image display unit 111, the user can qualitatively recognize the degree of recognition by the surgical tool recognizer.

Furthermore, the image display unit 111 is provided with a stability evaluation display unit 151 and a consistency evaluation display unit 152, and information indicating evaluation of quantitative stability between frames for the CG image 132 that is the recognition result, and information indicating evaluation of quantitative consistency between 3DCG models 141 and 141′ and the CG image 132 that is the recognition result, that is, quantitative evaluation of the degree of recognition by the surgical tool recognizer, are respectively displayed.

That is, in the case where the degree of recognition is insufficient, cross marks are attached to both the stability evaluation display unit 151 and the consistency evaluation display unit 152, and express that both are insufficient, as illustrated in the left part of FIG. 6.

In contrast, in the case where the degree of recognition is sufficient, check marks are attached to both a stability evaluation display unit 151′ and a consistency evaluation display unit 152′, and express that both are sufficient, as illustrated in the right part of FIG. 6.

With the display of the stability evaluation display units 151 and 151′ and the consistency evaluation display units 152 and 152′, the user can recognize the degree of recognition of the new surgical tool by the surgical tool recognizer on the basis of the quantitative evaluation.

Note that details of the stability evaluation and the consistency evaluation displayed by the stability evaluation display units 151 and 151′ and the consistency evaluation display units 152 and 152′ will be described below with reference to FIGS. 11 and 12, respectively.

Furthermore, the 3DCG models 141 and 141′ of the new surgical tool used for learning the surgical tool recognizer are displayed in the model display unit 112 in FIG. 6.

With the display of the model display unit 112, the user can compare and view the real image 131 of the surgical tool, the CG images 132 and 132′ as the recognition results, and the 3DCG models 141 and 141′ on the image display unit 111, and thus can qualitatively recognize the consistency between the current shapes of the 3DCG models 141 and 141′, the real image 131 of the actual surgical tool, and the CG images 132 and 132′ as the recognition results.

That is, in the case where the degree of recognition is insufficient, it is expressed that the 3DCG model 141 is thicker than and is different in shape from the real image 131, and the consistency is low, as illustrated in the left part of FIG. 6, for example.

In contrast, in the case where the degree of recognition is sufficient, it is expressed that the 3DCG model 141′ matches the shape of the real image 131, and the consistency is high, as illustrated in the right part of FIG. 3, for example.

The operation unit 55 is provided with the 3DCG model recreation button 55a operated when the 3DCG model is recreated, the learning termination button 55b operated when the learning is terminated, and the learning continuation button 55c operated when the learning is continued.

When the user operates the operation unit 55 according to display content of the image display unit 111 and the model display unit 112 in the feedback image displayed on the display 54, continuation of the learning, termination of the learning, or recreation of the 3DCG model of the new surgical tool recognizer can be executed.

That is, in the left part of FIG. 6, the real image 131 of the surgical tool and the CG image 132 as the recognition result are displayed in a shifted state, and the cross marks are attached to both the stability evaluation display unit 151 and the consistency evaluation display unit 152.

In the case of such display, the user can determine that the new surgical tool registered recognizer 95 cannot sufficiently recognize the new surgical tool, that is, the learning is insufficient, and thus operates, for example, the learning continuation button 55c.

Furthermore, in the model display unit 112 in the left part of FIG. 6, the 3DCG model 141 is expressed to be thicker than the real image 131 of the new surgical tool. In the case of such display, the user can determine that matching between the 3DCG model 141 of the new surgical tool and the real image 131 is not sufficient, and thus, for example, operates the 3DCG model recreation button 55a.

Therefore, the 3DCG model 141 is recreated on the basis of the current recognition result, and thus, is recreated with a more accurate shape and size. For example, as illustrated in the right part of FIG. 3, the learning data is newly created using the recreated 3DCG model 141′, and the learning of the surgical tool recognizer is continued.

Furthermore, since the feedback image is continuously displayed on the display 54 while the learning is continued, the state where the degree of recognition of the new surgical tool is improved by continuing the learning is presented in the feedback image.

Then, when the real image 131 of the new surgical tool and the CG image 132′ including the recognition result are superimposed and displayed in a matching state, and moreover, both the stability evaluation display unit 151 and the consistency evaluation display unit 152 are marked with the check marks to display information indicating appropriateness, and moreover, the 3DCG model 141′, the CG image 132, and the real image 131 are displayed with shapes appropriately matching each other, on the basis of the display content of the image display unit 111 and the model display unit 112 of the feedback image displayed on the display 54, as illustrated in the right part of FIG. 6, for example, it is regarded that sufficient learning has been performed enough to the state where the new surgical tool registered recognizer 95 can recognize the new surgical tool.

In such a case, the user operates the learning termination button 55b to terminate the learning, and updates and uses the learned recognizer 94 with the new surgical tool registered recognizer 95 learned for the new surgical tool.

As a result, in the case of updating the learned recognizer 94 so as to be able to recognize the new surgical tool, the user can update and use the learned recognizer 94 with the new surgical tool registered recognizer 95 after causing the new surgical tool registered recognizer 95 to learn enough to sufficiently recognize the new surgical tool.

### <Method of Generating 3DCG Model>

Next, a method of generating the 3DCG model will be described with reference to FIGS. 7 and 8.

The 3DCG model generation unit 71 includes the feature point matching processing unit 71a, and generates the three-dimensional point group information of the new surgical tool by performing the structure from motion processing by matching the feature points in a region of the surgical tool imaged from various angles continuously supplied from the laparoscope 31 between frames.

That is, the 3DCG model generation unit 71 forms three-dimensional point group information 171 of the feature points as illustrated in the right part of FIG. 7 by performing the structure from motion processing for each of the feature points forming a feature point group 161 indicated by the black circles in the drawing in a frame F1 while continuously performing matching up to a frame Fn, as illustrated in the left part of FIG. 7.

Furthermore, the 3DCG model generation unit 71 stores labeled template models of the 3DCG models in advance for a plurality of existing surgical tools.

Therefore, the 3DCG model generation unit 71 searches for the template model closest to the generated three-dimensional point group information 171 among the stored template models of the 3DCG model of the existing surgical tool by matching the three-dimensional point group information 171 generated by the feature point matching processing unit 71a with the stored template models of the 3DCG model of the existing surgical tool.

Then, the 3DCG model generation unit 71 adjusts the size of the searched template model of the 3DCG model with the generated three-dimensional point group information 171 by fitting to generate the 3DCG model of the new surgical tool.

At this time, the 3DCG model generation unit 71 simply labels the label attached to the searched template model in association with the 3DCG model of the new surgical tool.

That is, for example, a case where the template model of the 3DCG model having the shape closest to the three-dimensional point group information 171 generated in FIG. 7 is a template model 191 as illustrated in the left part of FIG. 8 will be considered.

In such a case, the 3DCG model generation unit 71 adjusts the searched template model 191 by fitting according to the size of the generated three-dimensional point group information 171 to deform the template model 191 to a template model 191m, as illustrated in the center of FIG. 8.

Then, the 3DCG model generation unit 71 sets the deformed template model 191m as the 3DCG model 91. At this time, the 3DCG model generation unit 71 simply labels a label 191a labeled in advance in the template model 191 as a label 91a of the generated 3DCG model 91.

Note that FIG. 8 illustrates that the label 191a indicating a movable fulcrum portion for the template model 191 including forceps is simply labeled as the label 91a of the 3DCG model 91. Furthermore, the label may be set in a manner other than this example, and various labels for the distal end and shafts are used as simple labels for the generated 3DCG model 91.

### <Learning>

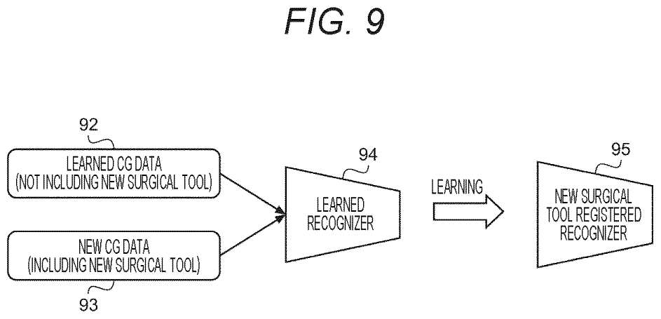

Next, an example of learning of the new surgical tool registered recognizer 95 using the learned CG data 92, the new CG data 93, and the learned recognizer 94 performed in the learning processing unit 82a of the surgical tool recognizer 82 in the learning data creation unit 52 will be described with reference to FIG. 9.

The learning processing unit 82a of the surgical tool recognizer 82 in the learning data creation unit 52 causes the new surgical tool registered recognizer 95 to learn by processing as illustrated in FIG. 9 using the learned CG data 92, the new CG data 93, and the learned recognizer 94 registered in the storage 53.

That is, the learning processing unit 82a generates the new surgical tool registered recognizer 95 (causes the new surgical tool registered recognizer 95 to learn) by adding the learning data including the new CG data 93 including the new surgical tool to the learning data including the learned CG data 92 not including the new surgical tool and initializing and learning a weight in the model of the learned recognizer 94.

Note that, when initializing the weight in the model of the learned recognizer 94, the learning processing unit 82a increases the number of dimensions in a final layer by the number of dimensions related to the new surgical tool.

By such learning, the learning processing unit 82a generates the new surgical tool registered recognizer 95 capable of recognizing the new surgical tool in addition to the surgical tool recognizable in the learned recognizer 94.

Then, when termination of the learning is instructed, the learning processing unit 82a updates the learned recognizer 94 with the new surgical tool registered recognizer 95, and updates the learned CG data 92 with all the CG data used in the learning so far, that is, information obtained by combining the original learned CG data 92 and the new CG data 93.

### <Recreation of 3DCG Model>

Next, the recreation of the 3DCG model will be described with reference to FIG. 10.

As illustrated in the left part of FIG. 10, in a case where the three-dimensional point group information 171 of the feature points includes a point group 171y and feature points 171x-1 to 171x-4 considered to be outliers, the template model of the 3DCG model having the closest shape is searched for on the basis of a distribution of the point group 171y and the feature points 171x-1 to 171x-4 considered to be outliers, and the size is adjusted.

Therefore, for example, as illustrated by the 3DCG model 141 in the model display unit 112 of the feedback image in the left part of FIG. 6, it is conceivable that the 3DCG model 141 becomes thicker than the real image 131 and does not match the real image 131.

In the recreation of the 3DCG model, the 3DCG model generation unit 71 researches for the template model on the basis of only the point 171y obtained by excluding the distribution of the feature points 171x-1 to 171x-4 considered to be outliers and not included within a predetermined distance from the frame of the CG image 132 as the current recognition result, that is, on the basis of the three-dimensional point group information narrowed down on the basis of the CG image 132 as the recognition result that is a search range, and fits the size with the point group 171y, thereby recreating the 3DCG model generation unit.

As a result, when the 3DCG model is recreated, the distribution of the feature points 171x-1 to 171x-4 considered to be outliers is excluded, so that an influence of the distribution of the feature points 171x-1 to 171x-4 considered to be outliers is eliminated. Therefore, the 3DCG model is recreated so as to approach the CG image 132 as the recognition result.

As a result, for example, as illustrated in the model display unit 112 of the feedback image in the right part of FIG. 6, the 3DCG model 141′ close to the real image 131 can be recreated.

### <Stability Evaluation>

Next, the stability evaluation will be described with reference to FIG. 11.

The stability evaluation is an index indicating how much the CG image serving as the recognition result by the surgical tool recognizer is continuously detected at a stable position.

That is, in the case where the learning is sufficiently performed and the degree of recognition by the surgical tool recognizer is high, the position of the CG image serving as the recognition result does not greatly change and is constant to some extent, or does not rapidly change even if there is a change.

On the other hand, in the case where the learning is insufficient and the degree of recognition by the surgical tool recognizer is low, the position of the CG image serving as the recognition result may greatly change, and the rapid change continues.

Therefore, the evaluation unit 73 obtains a mean value or a variance value in the magnitude of accelerations between consecutive frames at a predetermined position in the CG image serving as the recognition result as a quantitative stability evaluation value, and presents information corresponding to the stability evaluation value.

For example, as illustrated in FIG. 11, in the image display unit 111, the mean value or the variance value in the magnitude of the accelerations between frames of the CG image 132 serving as the recognition result in each of the consecutive frames F11 to F13 at time t1 to t3 is calculated in time series, and is set as the stability evaluation value.

That is, as illustrated in FIG. 11, a case where the CG image 132 at the time t1 moves to a CG image 132′ at the time t2, and further moves to a CG image 132″ at the time t3 will be considered.

Here, in the CG image 132, four feature points such as feature points 132a to 132d are set, and corresponding feature points are set to feature points 132a′ to 132d′ in the CG image 132′, and are set to feature points 132a″ to 132d″ in the CG image 132″.

Here, for example, the evaluation unit 73 calculates a velocity V1 between the feature points 132a and 132a′ from the distance the feature points and the time between the frames.

Furthermore, for example, the evaluation unit 73 calculates a velocity V2 between the feature points 132a′ and 132a″ from the distance the feature points and the time between the frames.

Then, the evaluation unit 73 calculates the acceleration of the feature point 132a among the three frames from a velocity difference (V2−V1) and the time among the frames.

Note that the method of obtaining the velocity and the acceleration is merely an example, and another method may be used.

The evaluation unit 73 similarly calculates the accelerations for the feature points 132b to 132d, obtains the respective mean values or the variance values of the accelerations of the feature points 132a to 132d, and sets the obtained values as the stability evaluation value that evaluates stability, and sets the stability evaluation value as an index indicating whether the learning is insufficient or sufficient by comparison with a predetermined value.

That is, in a case where the stability evaluation value including the mean values or the variance values of the accelerations of the feature points 132b to 132d is larger than the predetermined value, which can be regarded as having low stability, the evaluation unit 73 displays the cross mark on the stability evaluation display unit 151, for example, so as to indicate that the learning is insufficient. In this case, the learning may be continued without waiting for the determination of the user.

On the other hand, in a case where the stability evaluation value including the mean values or the variance values of the accelerations of the feature points 132b to 132d is smaller than the predetermined value, which can be regarded as having high stability, the evaluation unit 73 displays the check mark on the stability evaluation display unit 151, for example, so as to indicate that the learning is sufficient.

Note that, in the following description, processing of causing the learning to be continued without waiting for the determination by the user on the basis of the comparison between the stability evaluation value and the predetermined value will be described. However, even in a state where it is regarded that the learning is sufficient on the basis of the comparison between the stability evaluation value and the predetermined value and that continuation of the learning is unnecessary, the learning is continued when the learning continuation button 55c is operated according to determination by the user.

### <Consistency Evaluation>

Next, the consistency evaluation will be described with reference to FIG. 12.

The consistency evaluation is an index that evaluates how much the recognition result matches the 3DCG model, that is, the consistency between the recognition result and the 3DCG model.

For example, even in the case where it is determined that the stability evaluation value is smaller than the predetermined value and the learning is sufficient, if there is a deviation in the shape, size, or the like between the recognition result and the 3DCG model, the consistency is low and it cannot be said that the recognition result is appropriate. Furthermore, in the case where there is a deviation in the shape, size, or the like between the recognition result and the 3DCG model, it can be said that there is a deviation between the 3DCG model of the new surgical tool and the real image. That is, as the shapes and sizes of the recognition result and the 3DCG model match each other, the deviation between the 3DCG model of the new surgical tool and the real image is also smaller.

Therefore, the evaluation unit 73 calculates the consistency evaluation value as an index indicating how much the recognition result and the 3DCG model match each other by the comparison between the recognition result and the 3DCG model.

More specifically, in the image display unit 111, a distance Dr between the feature points 131a and 132b in the CG image 132 as the recognition result as illustrated in the left part of FIG. 12, and a distance Dm between feature points 91x and 91y in the 3DCG model 91 as illustrated in the corresponding right part of FIG. 12 are calculated, and a difference between the two, a ratio between the two, or the like, indicating the degree of coincidence between the two, is quantitatively calculated and is set as the consistency evaluation value.

Note that, in FIG. 12, both the CG image 132 as the recognition result and the 3DCG model 91 represent forceps, the feature points 132a and 91x are distal ends of the forceps, and the feature points 132b and 91y are movable fulcrum portions of the forceps.

When the evaluation unit 73 compares the consistency evaluation value with a predetermined value and determines that the consistency evaluation value is larger than the predetermined value, and the deviation between the recognition result and the 3DCG model is large, for example, the cross mark is displayed on the consistency evaluation display unit 152 so as to indicate that the recreation of the 3DCG model is necessary. In this case, the 3DCG model may be recreated without waiting for the determination of the user.

On the other hand, when the evaluation unit 73 compares the consistency evaluation value with the predetermined value and determines that the consistency evaluation value is smaller than the predetermined value, and the recognition result and the 3DCG model sufficiently match each other, for example, the check mark is displayed on the consistency evaluation display unit 152.

Note that, in the following description, processing of recreating the 3DCG model without waiting for the determination by the user on the basis of the comparison between the consistency evaluation value and the predetermined value will be described. However, even in a state where it is regarded that the learning is sufficient on the basis of the comparison between the consistency evaluation value and the predetermined value and that the 3DCG model is not recreated, the 3DCG model is recreated when the 3DCG model recreation button 55a is operated according to determination by the user.

### <New Surgical Tool Registration Processing>

Next, the new surgical tool registration processing by the surgical image display device 32 in FIG. 5 will be described with reference to the flowchart in FIG. 13.

In step S31, the new surgical tool registration unit 51 controls the display 54 to display an image prompting the laparoscope 31 to capture an image of the new surgical tool while changing the angle, and acquires an image in which the new surgical tool has been captured while continuously changing the angle captured by the laparoscope 31.

In step S32, the new surgical tool registration unit 51 controls the 3DCG model generation unit 71 to generate the 3DCG model on the basis of the image in which the new surgical tool has been captured. More specifically, the 3DCG model generation unit 71 controls the feature point matching processing unit 71a to detect the feature point in the region where the new surgical tool has been captured from the captured image, and acquires the three-dimensional point group information of the new surgical tool on the basis of continuous movement of the detected feature point.

In step S33, the 3DCG model generation unit 71 searches for the template model closest to the three-dimensional point group information of the new surgical tool among the stored template models of the 3DCG model.

In step S34, the 3DCG model generation unit 71 fits the template model of the 3DCG model closest to the three-dimensional point group information of the new surgical tool with the three-dimensional point group information, adjusts the size, and generates the 3DCG model corresponding to the new surgical tool.

In step S35, the 3DCG model generation unit 71 simply labels the generated 3DCG model with the label set to the template model searched when the 3DCG model is generated, and stores the generated 3DCG model as the 3DCG model 91 of the new surgical tool in the storage 53.

In step S36, the learning data creation unit 52 controls the simulator 81 to generate the CG data that is the learning data corresponding to the 3DCG model 91 of the new surgical tool stored in the storage 53. That is, the simulator 81 controls the 3DCG model reading unit 81a to read the 3DCG model 91 of the new surgical tool from the storage 53.

Furthermore, the simulator 81 controls the image creation unit 81b to generate the image of the operative field captured by the laparoscope 31, and combines the generated image with the read 3DCG model 91 of the new surgical tool to generate the CG data serving as the learning data by simulation, and stores the generated CG data as the new CG data 93.

In step S37, the surgical tool recognizer 82 controls the learning processing unit 82a to generate the new surgical tool registered recognizer 95 by learning. More specifically, the learning processing unit 82a reads the learned CG data 92, the new CG data 93, and the learned recognizer 94 stored in the storage 53, generates the new surgical tool registered recognizer 95 by learning by the method described with reference to FIG. 9, and stores the same in the storage 53.

In step S38, the new surgical tool registration unit 51 controls the surgical tool recognizer 72 to recognize the new surgical tool in the image captured by the laparoscope 31 by the recognition processing using the new surgical tool registered recognizer 95, and generates the image of the image display unit 111 in the feedback image in which the recognition result is superimposed and displayed on the real image of the new surgical tool in the image.

In step S39, the evaluation unit 73 calculates the stability evaluation value and the consistency evaluation value by the methods described with reference to FIGS. 11 and 12, compares each of the calculated stability evaluation value and consistency evaluation value with the predetermined value, and generates the evaluation result.

In step S40, the image generation unit 74 generates the image of the model display unit 112 on the basis of the 3DCG model 91, adds the stability evaluation display unit 151 and the consistency evaluation display unit 152 to the generated image of the recognition result from the evaluation results based on the stability evaluation value and the consistency evaluation value to generate the image display units 111, and combines them to generate the feedback image, and displays the feedback image on the display 54.

In step S41, the evaluation unit 73 determines whether or not the stability evaluation value is equal to or less than the predetermined value, and in a case where it is determined that the stability evaluation value is not equal to or less than the predetermined value and the learning is insufficient, the processing returns to step S36.

That is, it is determined whether or not the stability evaluation value is equal to or less than the predetermined value, and in the case where it is determined that the stability evaluation value is not equal to or less than the predetermined value and the learning is insufficient, the learning is continued by the processing of steps S36 to S41.

Then, in step S41, in a case where it is determined that the stability evaluation value is equal to or less than the predetermined value and the learning is sufficient, the processing proceeds to step S42.

In step S42, the evaluation unit 73 determines whether or not the consistency evaluation value is equal to or less than the predetermined value, and in a case where it is determined that the consistency evaluation value is not equal to or less than the predetermined value and the recreation of the 3DCG model is necessary, the processing proceeds to step S46.

In step S46, as described with reference to FIG. 10, the 3DCG model generation unit 71 narrows down the search range of the three-dimensional point group information on the basis of the recognition result.

Then, the processing returns to step S33, and the 3DCG model generation unit 71 recreates the 3DCG model on the basis of the three-dimensional point group information in which the search range is narrowed down on the basis of the recognition result, and the learning is continued.

That is, in step S41, in the case where it is determined that the recreation of the 3DCG model is necessary, the processing in steps S46 and S33 to S41 is repeated to recreate the 3DCG model, and the learning using the recreated 3DCG model is repeated.

Then, in step S41, in the case where it is determined that the consistency evaluation value is equal to or less than the predetermined value and the recreation of the 3DCG model is not necessary, the processing proceeds to step S43.

In step S43, the new surgical tool registration unit 51 determines whether or not the learning termination button 55b has been operated, and in the case where the learning termination button has not been operated, the processing proceeds to step S44.

In step S44, the new surgical tool registration unit 51 determines whether or not the learning continuation button 55c has been operated, and in the case where the learning continuation button has not been operated, the processing proceeds to step S45.

In step S45, the learning data creation unit 52 determines whether or not the 3DCG model recreation button 55a has been operated, and in the case where the learning data creation unit has not been operated, the processing returns to step S41.

That is, in the case where none of the 3DCG model recreation button 55a, the learning termination button 55b, and the learning continuation button 55c of the operation unit 55 is not operated by the processing in steps S43 to S45, the processing in steps S41 to S45 is continued, and the state where the feedback image is displayed is continued.

In step S45, in the case where the user has operated the 3DCG model recreation button 55a on the basis of the feedback image, the processing proceeds to step S46, the 3DCG model is recreated, and the learning based on the recreated 3DCG model is continued.

Furthermore, in step S44, in the case where the user has operated the learning continuation button 55c on the basis of the feedback image, the processing returns to step S36, and the learning is continued.

Then, in step S43, in the case where the user has operated the learning termination button 55b on the basis of the feedback image, the processing proceeds to step S47.

In step S47, the new surgical tool registration unit 51 updates the learned recognizer 94 with the generated new surgical tool registered recognizer 95 and adds the new CG data 93 to the learned CG data 92 to update the learned CG data 92, and terminates the processing.

That is, by the above-described series of processing, the 3DCG model 91 of the new surgical tool is generated by capturing the image of the new surgical tool while changing the angle, the new CG data 93 that is the learning data is generated using the generated 3DCG model, the new surgical tool registered recognizer 95 is generated on the basis of the learning using the generated new CG data 93, the learned CG data 92, and the learned recognizer 94, the new surgical tool is recognized by the generated new surgical tool registered recognizer 95, the evaluation value based on the recognition result is calculated, and the learning is continued or the 3DCG model is recreated and the learning is continued on the basis of the evaluation result.

At this time, since the 3DCG model, the recognition result, and the evaluation value are displayed as the feedback image, the user can continue the learning or recreate the 3DCG model on the basis of the 3DCG model, the recognition result, and the evaluation value by himself/herself on the basis of the feedback image.

As a result, when registering the new surgical tool in the state of being recognizable manner, the user causes the laparoscope 31 to capture the image of the new surgical tool while changing the angle in the surgical environment, and executes the above-described series of new surgical tool registration processing, and updates the learned recognizer 94 after confirming that the new surgical tool is sufficiently recognizable, and uses the learned recognizer 94 in the actual surgery in the surgical image display processing unit 50.

Note that, in the above, an example has been described in which the new surgical tool registration processing is performed by the new surgical tool registration unit 51, the learning data creation unit 52, the storage 53, the display 54, and the operation unit 55 in the surgical image display device 32. However, each of the new surgical tool registration unit 51, the learning data creation unit 52, and the storage 53 may be implemented by a server computer or a cloud computer on a network.

### 3. Modification of New Surgical Tool Registration Processing

In the above, an example has been described in which the new surgical tool registered recognizer 95 is caused to learn by generating the 3DCG model by capturing the image of the new surgical tool while changing the angle and then generating the CG data that is the learning data before starting the surgery.

However, in a case where it is felt that the learning is insufficient after the operation is started after the new surgical tool is registered once, the processing of generating the 3DCG model may be omitted since the 3DCG model corresponding to the new surgical tool has already been generated.

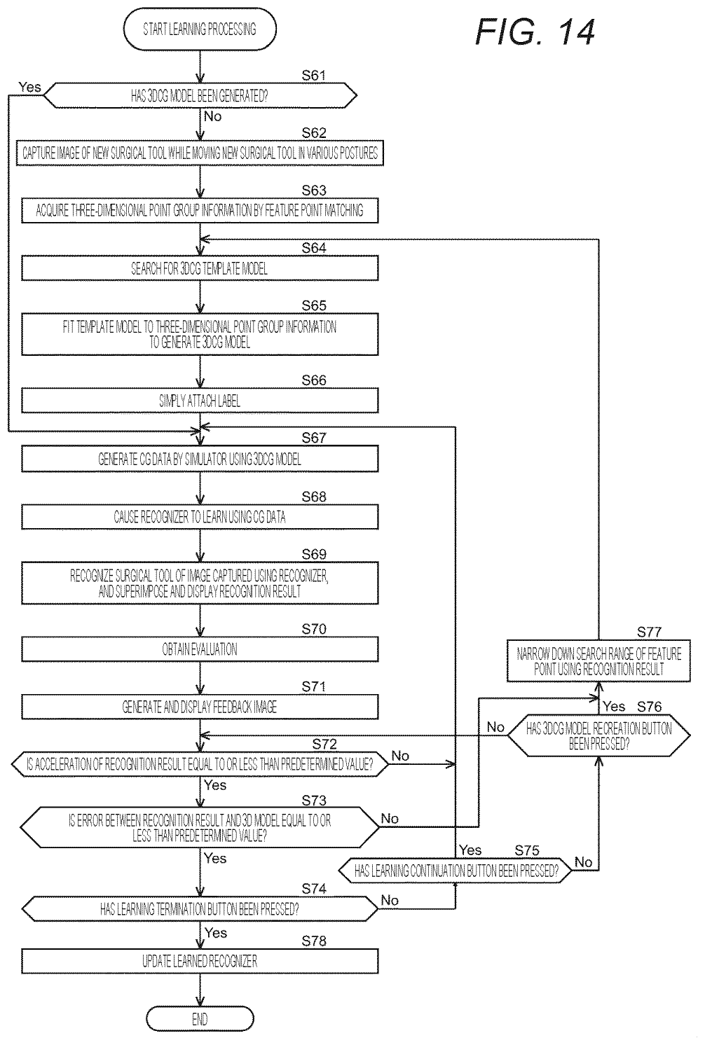

Therefore, a modification of the new surgical tool registration processing in which the processing of generating the 3DCG model is omitted when the 3DCG model has already been generated will be described with reference to the flowchart of FIG. 14.

Note that the processing in steps S62 to S78 in the flowchart in FIG. 14 is similar to the processing in steps S31 to S47 in FIG. 13, and therefore description thereof will be omitted.

That is, in step S61, the new surgical tool registration unit 51 determines whether or not the 3DCG model 91 has been registered in the storage 53.

In step S61, in a case where it is determined that the 3DCG model 91 has not been registered in the storage 53, the processing proceeds to step S62, and an image of the new surgical tool is captured while changing the angle, and the 3DCG model 91 corresponding to the new surgical tool is generated.

On the other hand, in step S61, in the case where it is determined that the 3DCG model 91 has been registered in the storage 53, the processing in steps S62 to S66 is skipped, and the learning of the new surgical tool registered recognizer 95 using the 3DCG model is started.

According to the above processing, when it is desired to continue the learning again or to recreate the 3DCG model after the processing of registering the new surgical tool is once performed, it is possible to omit the processing of capturing the image of the new surgical tool and creating the 3DCG model 91. Therefore, it is possible to more easily continue the learning of the new surgical tool and improve the recognition accuracy.

Furthermore, at this time, even in the case where the 3DCG model 91 is recreated, it is possible to generate the 3DCG model 91 more similar to the new surgical tool after narrowing down the three-dimensional point group information using the recognition result, and it is possible to realize more highly accurate learning.

### 4. Example of Execution by Software

Incidentally, the series of processing described above can be executed by hardware, but can also be executed by software. In a case where the series of processing is performed by software, a program constituting the software is installed from a recording medium into, for example, a computer built into dedicated hardware or a general-purpose computer that is capable of performing various functions by installing various programs, or the like.

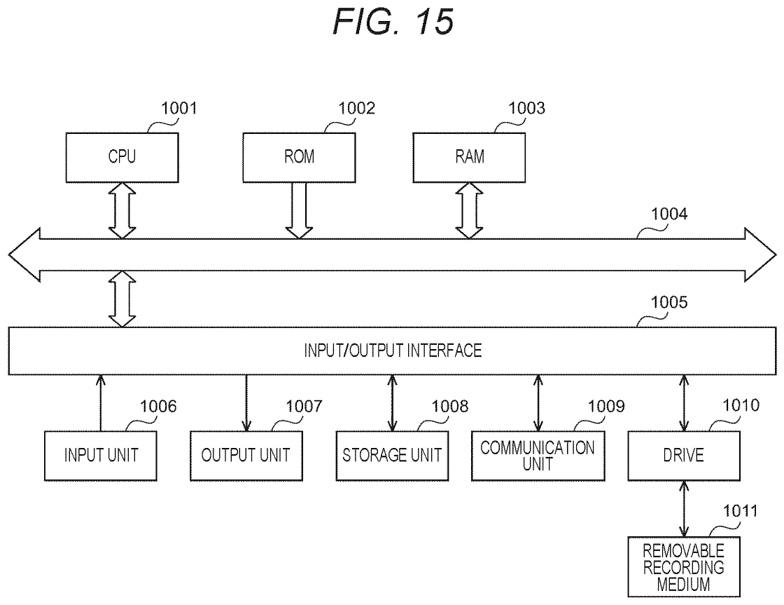

FIG. 15 illustrates a configuration example of a general-purpose computer. This computer includes a central processing unit (CPU) 1001. An input/output interface 1005 is connected to the CPU 1001 via a bus 1004. A read only memory (ROM) 1002 and a random access memory (RAM) 1003 are connected to the bus 1004.

To the input/output interface 1005, an input unit 1006 including an input device such as a keyboard and a mouse by which a user inputs operation commands, an output unit 1007 that outputs a processing operation screen and an image of a processing result to a display device, a storage unit 1008 that includes a hard disk drive and the like and stores programs and various data, and a communication unit 1009 including a local area network (LAN) adapter or the like and performs communication processing via a network represented by the Internet are connected. Furthermore, a drive 1010 that reads and writes data from and to a removable storage medium 1011 such as a magnetic disk (including flexible disk), an optical disc (including compact disc-read only memory (CD-ROM) and digital versatile disc (DVD)), a magneto-optical disk (including mini disc (MD)), or a semiconductor memory is connected.

The CPU 1001 performs various processing in accordance with a program stored in the ROM 1002, or a program read from the removable storage medium 1011 such as a magnetic disk, an optical disc, a magneto-optical disk, or a semiconductor memory, installed in the storage unit 1008, and loaded from the storage unit 1008 into the RAM 1003. Furthermore, the RAM 1003 also appropriately stores data necessary for the CPU 1001 to perform various processing, and the like.

In the computer configured as described above, for example, the CPU 1001 loads the program stored in the storage unit 1008 into the RAM 1003 via the input/output interface 1005 and the bus 1004 and executes the program, to thereby perform the above-described series of processing.

The program executed by the computer (CPU 1001) can be provided by being recorded in the removable storage medium 1011 as a package medium or the like, for example. Furthermore, the program can be provided via a wired or wireless transmission medium, such as a local area network, the Internet, or digital satellite broadcasting.

In the computer, the program can be installed in the storage unit 1008 via the input/output interface 1005 by attaching the removable storage medium 1011 to the drive 1010. Furthermore, the program can be received by the communication unit 1009 via a wired or wireless transmission medium and installed in the storage unit 1008. In addition, the program can be installed in the ROM 1002 or the storage unit 1008 in advance.

Note that the program executed by the computer may be a program that executes processing in time series in the order described in the present description, or a program that executes processing in parallel or at a necessary timing such as when a call is made.

Note that the CPU 1001 in FIG. 15 implements the functions of the new surgical tool registration unit 51 and the learning data creation unit 52 in FIG. 5, the storage unit 1008 implements the function of the storage 53 in FIG. 5, and the input unit 1006 implements the function of the operation unit 55 in FIG. 5.

Furthermore, in the present description, a system is intended to mean assembly of a plurality of components (devices, modules (parts), and the like) and it does not matter whether or not all the components are in the same casing. Therefore, a plurality of devices accommodated in separate casings and connected via a network and one device in which a plurality of modules is accommodated in one casing are both systems.

Note that embodiments of the present disclosure are not limited to the embodiments described above, and various modifications may be made without departing from the scope of the present disclosure.

For example, the present disclosure may have a configuration of cloud computing in which one function is shared by a plurality of devices via a network and processing is performed in cooperation.

Furthermore, each of the steps described in the above-described flowcharts can be executed by one device or executed by a plurality of devices in a shared manner.

Moreover, in a case where a plurality of pieces of processing is included in one step, the plurality of pieces of processing included in the one step can be executed by one device or executed by a plurality of devices in a shared manner.

Note that the present disclosure may have the following configurations.

<1>

An information processing device including:
a computer graphics (CG) data generation unit configured to generate, on a basis of an image including a new surgical tool, CG data serving as learning data to be used to learn a recognizer that recognizes the new surgical tool;
a learning unit configured to cause the recognizer to learn, the recognizer recognizing the new surgical tool, on a basis of the CG data; and
an evaluation unit configured to calculate an evaluation value that evaluates a recognition result of the new surgical tool by the recognizer based on the image, in which
the learning unit continues the learning of the recognizer according to the evaluation value.
   <2>

The information processing device according to <1>, in which
the evaluation unit calculates an inter-frame acceleration of a recognition position of the recognition result of the new surgical tool by the recognizer as a stability evaluation value, and
the learning unit continues the learning of the recognizer according to the stability evaluation value.
   <3>

The information processing device according to <2>, in which
the learning unit continues the learning of the recognizer when the stability evaluation value is larger than a predetermined value.
   <4>

The information processing device according to <1>, further including:
a display unit configured to generate and display a feedback image in which a CG image corresponding to the recognition result of the new surgical tool is superimposed and displayed on the new surgical tool in the image.
   <5>

The information processing device according to <4>, in which,
in the feedback image, the computer graphics (CG) image corresponding to the recognition result of the new surgical tool is superimposed and displayed on the new surgical tool in the image, and information indicating necessity of continuation of the learning of the recognizer is displayed according to the evaluation value.
   <6>

The information processing device according to <5>, further including:
a learning continuation instruction operation unit operated by a user when instructing continuation of the learning of the recognizer, in which,
when the learning continuation instruction operation unit is operated, the learning unit continues the learning of the recognizer.
   <7>

The information processing device according to <5>, further including:
a learning termination instruction operation unit operated by a user when instructing termination of the learning of the recognizer, in which,
when the learning termination instruction operation unit is operated, the learning unit terminates the learning of the recognizer.
   <8>

The information processing device according to <1>, further including:
a three-dimensional computer graphics (3DCG) model generation unit configured to generate a 3DCG model of the new surgical tool on the basis of the image, in which
the CG data generation unit generates the CG data serving as the learning data to be used to learn the recognizer that recognizes the new surgical tool, using the 3DCG model.
   <9>

The information processing device according to <8>, in which
the 3DCG model generation unit generates three-dimensional point group information of the new surgical tool on the basis of the image, searches for a template model closest to the three-dimensional point group information among template models of 3DCG models of a plurality of surgical tools stored in advance, and fits the searched template model on the basis of the three-dimensional point group information to generate the 3DCG model.
   <10>

The information processing device according to <9>, in which
the evaluation unit calculates a difference between the 3DCG model and the recognition result of the new surgical tool by the recognizer as a consistency evaluation value, and
the 3DCG model generation unit recreates the 3DCG model according to the consistency evaluation value.
   <11>

The information processing device according to <10>, in which
the 3DCG model generation unit recreates the 3DCG model when the consistency evaluation value is larger than a predetermined value.
   <12>

The information processing device according to <10>, in which
the 3DCG model generation unit recreates the 3DCG model, using the three-dimensional point group information narrowed down on the basis of the recognition result of the new surgical tool, of the three-dimensional point group information used in creating the 3DCG model, when recreating the 3DCG model.
   <13>

The information processing device according to <8>, further including:
a display unit configured to generate and display a feedback image in which a CG image corresponding to the recognition result of the new surgical tool is superimposed and displayed and the 3DCG model is displayed on the new surgical tool in the image.
   <14>

The information processing device according to <13>, in which,
in the feedback image, the computer graphics (CG) image corresponding to the recognition result of the new surgical tool is superimposed and displayed, the 3DCG model is displayed, and information indicating necessity of recreation of the 3DCG model is displayed according to the evaluation value, on the new surgical tool in the image.
   <15>

The information processing device according to <14>, further including:
a recreation instruction operation unit operated by a user when instructing recreation of the 3DCG model, in which,
when the recreation instruction operation unit is operated, the 3DCG model generation unit recreates the 3DCG model.
   <16>

The information processing device according to any one of <1> to <15>, in which
the image is an image captured by a laparoscope.
   <17>

An information processing method including steps of:
generating, on a basis of an image including a new surgical tool, computer graphics (CG) data serving as learning data to be used to learn a recognizer that recognizes the new surgical tool;
causing the recognizer to learn, the recognizer recognizing the new surgical tool, on a basis of the CG data; and
calculating an evaluation value that evaluates a recognition result of the new surgical tool by the recognizer based on the image, in which
the learning of the recognizer is continued according to the evaluation value.
   <18>

A program for causing a computer to function as:
a computer graphics (CG) data generation unit configured to generate, on a basis of an image including a new surgical tool, CG data serving as learning data to be used to learn a recognizer that recognizes the new surgical tool;
a learning unit configured to cause the recognizer to learn, the recognizer recognizing the new surgical tool, on a basis of the CG data; and
an evaluation unit configured to calculate an evaluation value that evaluates a recognition result of the new surgical tool by the recognizer based on the image, in which
the learning unit continues the learning of the recognizer according to the evaluation value.

### REFERENCE SIGNS LIST

11 Surgical image display system
31 Laparoscope
32 Surgical image display device
41 RGB camera
50 Surgical image display processing unit
51 New surgical tool registration unit
52 Learning data creation unit
53 Storage
54 Display
55 Operation unit
55a 3DCG model creation button
55b Learning termination button
55c Learning continuation button
61 Surgical tool recognizer
61a Inference unit
62 Image generation unit
71 3DCG model generation unit
71a Feature point matching processing unit
72 Surgical tool recognizer
72a Inference unit
73 Evaluation unit
74 Image generation unit
81 Simulator
81a 3DCG model reading unit
81b Image creation unit
82 Surgical tool recognizer
82a Learning processing unit
91 3DCG model
92 Learned CG data
93 New CG data
94 Learned recognizer
95 New surgical tool registered recognizer

1. An information processing device comprising:
a computer graphics (CG) data generation unit configured to generate, on a basis of an image including a new surgical tool, CG data serving as learning data to be used to learn a recognizer that recognizes the new surgical tool;
a learning unit configured to cause the recognizer to learn, the recognizer recognizing the new surgical tool, on a basis of the CG data; and
an evaluation unit configured to calculate an evaluation value that evaluates a recognition result of the new surgical tool by the recognizer based on the image, wherein
the learning unit continues the learning of the recognizer according to the evaluation value.

2. The information processing device according to claim 1, wherein
the evaluation unit calculates an inter-frame acceleration of a recognition position of the recognition result of the new surgical tool by the recognizer as a stability evaluation value, and
the learning unit continues the learning of the recognizer according to the stability evaluation value.

3. The information processing device according to claim 2, wherein
the learning unit continues the learning of the recognizer when the stability evaluation value is larger than a predetermined value.

4. The information processing device according to claim 1, further comprising:
a display unit configured to generate and display a feedback image in which a CG image corresponding to the recognition result of the new surgical tool is superimposed and displayed on the new surgical tool in the image.

5. The information processing device according to claim 4, wherein,
in the feedback image, the computer graphics (CG) image corresponding to the recognition result of the new surgical tool is superimposed and displayed on the new surgical tool in the image, and information indicating necessity of continuation of the learning of the recognizer is displayed according to the evaluation value.

6. The information processing device according to claim 5, further comprising:
a learning continuation instruction operation unit operated by a user when instructing continuation of the learning of the recognizer, wherein,
when the learning continuation instruction operation unit is operated, the learning unit continues the learning of the recognizer.

7. The information processing device according to claim 5, further comprising:
a learning termination instruction operation unit operated by a user when instructing termination of the learning of the recognizer, wherein,
when the learning termination instruction operation unit is operated, the learning unit terminates the learning of the recognizer.

8. The information processing device according to claim 1, further comprising:
a three-dimensional computer graphics (3DCG) model generation unit configured to generate a 3DCG model of the new surgical tool on the basis of the image, wherein
the CG data generation unit generates the CG data serving as the learning data to be used to learn the recognizer that recognizes the new surgical tool, using the 3DCG model.

9. The information processing device according to claim 8, wherein
the 3DCG model generation unit generates three-dimensional point group information of the new surgical tool on the basis of the image, searches for a template model closest to the three-dimensional point group information among template models of 3DCG models of a plurality of surgical tools stored in advance, and fits the searched template model on the basis of the three-dimensional point group information to generate the 3DCG model.

10. The information processing device according to claim 9, wherein
the evaluation unit calculates a difference between the 3DCG model and the recognition result of the new surgical tool by the recognizer as a consistency evaluation value, and
the 3DCG model generation unit recreates the 3DCG model according to the consistency evaluation value.

11. The information processing device according to claim 10, wherein
the 3DCG model generation unit recreates the 3DCG model when the consistency evaluation value is larger than a predetermined value.

12. The information processing device according to claim 10, wherein
the 3DCG model generation unit recreates the 3DCG model, using the three-dimensional point group information narrowed down on the basis of the recognition result of the new surgical tool, of the three-dimensional point group information used in creating the 3DCG model, when recreating the 3DCG model.

13. The information processing device according to claim 8, further comprising:
a display unit configured to generate and display a feedback image in which a CG image corresponding to the recognition result of the new surgical tool is superimposed and displayed and the 3DCG model is displayed on the new surgical tool in the image.

14. The information processing device according to claim 13, wherein,
in the feedback image, the computer graphics (CG) image corresponding to the recognition result of the new surgical tool is superimposed and displayed, the 3DCG model is displayed, and information indicating necessity of recreation of the 3DCG model is displayed according to the evaluation value, on the new surgical tool in the image.

15. The information processing device according to claim 14, further comprising:
a recreation instruction operation unit operated by a user when instructing recreation of the 3DCG model, wherein,
when the recreation instruction operation unit is operated, the 3DCG model generation unit recreates the 3DCG model.

16. The information processing device according to claim 1, wherein
the image is an image captured by a laparoscope.

17. An information processing method comprising steps of:
generating, on a basis of an image including a new surgical tool, computer graphics (CG) data serving as learning data to be used to learn a recognizer that recognizes the new surgical tool;
causing the recognizer to learn, the recognizer recognizing the new surgical tool, on a basis of the CG data; and
calculating an evaluation value that evaluates a recognition result of the new surgical tool by the recognizer based on the image, wherein
the learning of the recognizer is continued according to the evaluation value.

18. A program for causing a computer to function as:
a computer graphics (CG) data generation unit configured to generate, on a basis of an image including a new surgical tool, CG data serving as learning data to be used to learn a recognizer that recognizes the new surgical tool;
a learning unit configured to cause the recognizer to learn, the recognizer recognizing the new surgical tool, on a basis of the CG data; and
an evaluation unit configured to calculate an evaluation value that evaluates a recognition result of the new surgical tool by the recognizer based on the image, wherein
the learning unit continues the learning of the recognizer according to the evaluation value.
